(19) United States
(12) Patent Application Publication
(10) Pub. No.: US 2011/0208279 A1
(43) Pub. Date: Aug. 25, 2011

(54) Method and therapeutic apparatus for normalizing function of sinus cilia using heat

(76) Inventor: Daniel Allen Sanker, Fayetteville, AR (US)

(21) Appl. No.: 12/709,111

(22) Filed: Feb. 19, 2010

(51) Int. Cl. A61F 7/08

(52) U.S. Cl. 607/109

(57) ABSTRACT

The invention relates to a non-surgical and non-invasive drug-free therapeutic apparatus and method of using thermal energy-in-transfer to normalize poor sinus cilia function that can cause upper respiratory tract problems. The nasal and sinus cavity membranes have thousands of very small hair-like organelles called cilia. They are capable of rhythmic beating like oars, and they keep the sinus cavities clear of debris, pollutants and excessive drainage. This Mucociliary Clearance System performs best at a temperature slightly above the human body's normothermia. The method and therapeutic apparatus includes a mask with a pocket cavity that accommodates a heating element. The heating element can use heat from a disposable heating pack. When the apparatus is affixed to the human head with the heating element engaged, the warmth helps restore normal cilia function by bringing the temperature of the sinus cavities to a more optimal temperature which is above normothermia.

### REFERENCES CITED

U.S. Patent Documents

| | | |
|---|---|---|
| 1,743,801 | January 1930 | Reynolds |
| 1,917,117 | July 1933 | Hines |
| 1,924,315 | August 1933 | Hemphill et al. |
| 2,191,937 | February 1940 | Low |
| 2,305,080 | December 1942 | Hemphill |
| 2,342,840 | February 1944 | Cadous |
| 2,671,898 | March 1954 | Wade |
| 2,716,981 | September 1955 | More |
| 2,891,252 | June 1959 | Lazo |
| 2,946,133 | July 1960 | Williams |
| 4,425,917 | January 1984 | Kuznetz |
| 4,512,830 | April 1985 | Hulett et al. |
| 4,532,410 | July 1985 | Wehmeyer |
| 4,612,934 | September 1986 | Borkan |
| 4,635,625 | January 1987 | Teeple |
| 4,649,908 | March 1987 | Ghaly |
| 4,676,247 | June 1987 | Van Cleve |
| 4,688,572 | August 1987 | Hubbard et al. |
| 4,854,319 | November 1987 | Tobi |
| 4,872,217 | March 1988 | Kitayama |
| 4,815,144 | March 1989 | Martin |
| 4,848,334 | July 1989 | Bellm |
| 4,972,832 | November 1990 | Trapini et al. |
| 5,020,711 | June 1991 | Kelley |
| 5,038,779 | August 1991 | Barry et al. |
| 5,072,598 | December 1991 | Dibrell |
| 5,088,549 | February 1992 | Schneider |
| 5,148,804 | September 1992 | Hill et al. |
| 5,146,625 | September 1992 | Steele et al. |
| 5,190,036 | March 1993 | Linder |
| 5,230,333 | July 1993 | Yates et al. |
| 5,197,292 | March 1993 | McPherson |
| 5,295,949 | March 1994 | Hathaway |
| 5,305,470 | April 1994 | McKay |
| 5,605,144 | April 1994 | Simmons |
| 5,395,400 | March 1995 | Stafford et al. |
| 5,484,448 | January 1996 | Steele et al. |
| 6,019,101 | October 1996 | Cotner |
| 5,940,886 | January 1998 | McCarthy Smith |
| 6,537,308 | April 2000 | Burkhart |
| 6,237,154 | May 2000 | Reuven |
| 6,792,624 | January 2002 | Simmons |
| 6,411,852 | June 2002 | Danek |
| 6,415,183 | July 2002 | Scheiner |
| 6,745,397 | October 2004 | Magidson |
| 7,461,656 | November 2006 | Gunaratnam |
| 7,469,162 | December 2008 | Lattner |

### OTHER REFERENCES

Dr. Dailley; “Sinus Disease, What is Sinus Disease and what causes the condition?” Center for Breath Treatment; 2000; accessed online Dec. 2, 2009: http://www.breathcure.com/v/sinus/sinus-disease.html.
“Sinus Disease”; Jan. 21, 2001; Dr. Grossan's Ear, Nose and Throat Consultant Pages; accessed online Dec. 2, 2009: http://www.ent-consult.com/sinus_diseasepro.html.
M. Jorissen and A. Bessems; “European Archives of Oto-Rhino-Laryngology”; April 1995; Abstract accessed online Dec. 2, 2009: http://www.springerlink.com/content/ht3025421tn4gwr1/.
Doctor Tbalu; “Rhinology—Pathophysiology of Sinusitis”; Jun. 10, 2008; accessed online Dec. 2, 2009: http://sites.google.com/site/drtbalusotolaryngology/rhinology/pathophysiology-of-sinusitis.
Hesse, Mizera, Kasparek, & Konietzko; Article in German, translated by Klin Wochenschr; February, 1981; “Method of Photometric Measurement of Ciliary Beat Frequency from Human Tracheobronchial Epithelium”; accessed online Dec. 2, 2009: http://www.ncbi.nlm.nih.gov/pubmed/7206602.

### DESCRIPTION

1. Field of the Invention

This invention relates to an apparatus for improving sinus cilia function with an appliance that allows transference of heat through the face to the sinus cilia.

2. Background of the Invention

The nasal and sinus cavity membranes have thousands of very small hair-like organelles called cilia. They are capable of rhythmic beating like oars, and they keep the sinus cavities clear and clean from debris, pollutants and excessive drainage.

When they are pulsating according to their normal rhythm, they move the bacteria from front to back of the nose. This system is called The Mucociliary Clearance System which consists of the cells of the surface of the nose, cilia and mucus. The system operates best if it is brought to a temperature slightly above normal human body temperature and is relatively known as normothermia. If the Mucociliary Clearance System is performing abnormally, problems that would arise may include: sinus irritation, sinus infection or sinusitis which is defined as the inflammation of the sinuses and nasal passages along with headache or pressure in the eyes, nose, cheek area or on one side of the head. A person with a sinus problem may also experience having coughs, fever, bad breath, and nasal congestion with thick nasal secretions. The method and therapeutic apparatus includes a mask with a pocket cavity that accommodates a heating element. The heating element can use heat from a disposable thermal hot pack—a gel pack that may be temporarily heated by an external source. When the apparatus is affixed to the human head with the heating element engaged, the warmth helps restore normal cilia function by bringing the temperature of the sinus cavities to a more optimal temperature which is above normothermia. Cilia are positively activated by increasing the environmental temperature not above 104 degrees Fahrenheit. Up until now this has been accomplished with warm water-based sinus irrigators or sinus douches which are inconvenient and have side effects associated with shooting water up the human nostrils. The water and its associated pressure are harsh on the sinuses and can cause side effects. In addition, it is inconvenient to use water to transfer heat into the sinus passages.

The nose and sinuses form the first part of the respiratory system, the body system that exchanges gases between air and blood. The nose is the main organ that allows air to flow into the lungs. Air enters the body through two passageways called the nostrils. Each nostril opens up into its own nasal cavity (large spaces inside the front of the face).

The most important function of the nose and nasal cavity is to process each breath before it enters the lungs. This includes:
Filtering out dangerous particles (e.g., bacteria, viruses, dust and pollen)
Warming each breath to prevent cold air from damaging sensitive lung tissues
Adding moisture to each breath to prevent airways and lungs from drying out

In addition, the nose is part of the olfactory system, which is used for smelling.

The sinuses are air-filled pockets found within the bones of the nose and face. The sinuses appear in pairs on either side of the face. There are four different types of sinuses, depending in type and in varying sizes of tiny up to the size of a walnut. The precise role of the sinuses remains unclear, although they are believed to serve several functions, including:
Reducing the weight of the skull while preserving bone strength and shape
Adding resonance to the voice
Reducing damage sustained during a head trauma by absorbing some of the impact

The nose, nasal cavity and sinuses are the first few components of the respiratory system. The respiratory system is made up of the pharynx (the passageway at the back of the throat), larynx (voice box), trachea (windpipe), bronchi (bronchial tubes) and lungs.

The nose is the main organ that allows air to flow into the lungs. For most adults, about 18,000 to 20,000 liters of air pass through the nose every day. The nose offers an entrance to the respiratory system (the body system that exchanges gases between air and blood), through the two passageways (nostrils). The structure supporting the upper part of the external nose is made of bone, while the lower part is made of cartilage.

Each nostril opens up into its own nasal cavity, which is separated by the nasal septum. The septum is made of bone and cartilage and runs from the nostrils to the back of the throat. Bones that project into the nasal cavity (nasal conchae) form a series of folds (turbinates) that increase the surface area of each nasal cavity. The back of the nasal cavity opens up into the airways that lead to the lungs. The nose also acts as the main passageway for air to leave the body when a person exhales. Air can also enter and leave the body through the mouth or the oral cavity.

The most important function of the nose and nasal cavity is to process each breath before it enters the lungs. This filtering process is important for several reasons. Considering the amount of air that passes through the respiratory system each day, it is important that the nose:
Filter out dangerous particles (e.g., bacteria, viruses, dust and pollen) that could otherwise enter the lungs which may cause damage. Tiny hairs (cilia) along with mucous membranes line the inside of the nose to trap particles before they enter the body. The cilia are capable of small movements that can direct the flow of mucus (a substance secreted by mucous membranes), removing it from the nasal cavity. Sneezing forces air through the nasal cavity and is also effective in removing particles and mucus.
Warm each breath to prevent cold air from damaging sensitive lung tissues. The large amount of surface area and many blood vessels in the nasal cavity help warm each breath quickly as heat transfers from the blood to the passing air.
Add moisture to each breath to prevent the airways and lungs from becoming dry and damaged. As air passes through the nasal cavity, moisture is transferred from the mucus secreted by the lining of the nasal cavity to the air.

The nose and nasal cavity are also important for several reasons:
The nasal cavity contains smell receptor cells that signal the brain upon encountering different chemicals. The brain can interpret these signals as various smells. The sense of smell also affects a person's perception of the taste of foods. When people sniff, air flow is increased over the smell receptor cells. This increases a person's odor exposures.
Tears drain into the nasal cavity from the eyes when crying. This drainage occurs through the tear duct known as the nasolacrimal duct. This drainage helps prevent foreign particles from entering the nose and explains why a person's nose “runs” when crying.
The eustachian tube runs from the back of the nasal cavity to the ears, equalizing the pressure between them. This connection also allows for ear drainage and ventilation, which helps prevent infection.
The nasal cavity is connected to several of the sinuses, allowing for drainage from these areas. This allows for the easy removal of foreign bacteria and viruses from the sinuses, which helps prevent infection.

At the back of the nasal cavity is the nasopharynx, which forms the upper part of the throat. The nasopharynx contains adenoid tissues. The adenoid is made up of lymph tissues, which plays a vital role in fighting infections during the first year of life.

Common colds and other infections can cause inflammation of the nasopharynx, This can block the eustachian tubes, which results in a feeling of clogged ears.

Patients experiencing prolonged or recurrent nasal problems, such as congestion, sneezing, runny nose or thick nasal discharge are encouraged to contact their physician. These symptoms may require prompt medical attention because they can aggravate lung problems and lead to other conditions. For example, untreated nasal allergies can increase a person's risk of developing asthma (a chronic inflammation of the airway tissues) and make existing asthma worse.

The sinuses are hollow spaces located in the face and skull. They appear in pairs on either side of the face. Depending on the type, the sinuses vary in size from tiny to the size of a walnut. The four sets of sinuses are:
Frontal sinuses. Located in the forehead. There are two of these sinuses, one per side of the forehead. The frontal sinuses vary greatly from person to person both in size and shape.
Maxillary sinuses. Located in the cheeks, between the teeth and the eyes. There are two of these sinuses, one in each cheek. These are the largest of the sinuses, and each can roughly be as large as a walnut.
Ethmoid sinuses. Located between the eyes, on each side of the nose. There are between 6 and 12 of these sinuses on each side of the face. These sinuses are very small.
Sphenoid sinuses. Located deep beneath the eyes, towards the middle of the skull. There are two of these sinuses, one per side. The size, shape and volume of these sinuses vary greatly from person to person.

Like the nasal cavity, the sinuses are lined with mucous membranes—moist layers of tissue that secrete mucus. These mucus membranes help remove foreign particles that enter the sinuses. The sinuses also have cilia, tiny hairs which push the mucus back into the nasal cavity through small openings (ostia). This drainage is not based on gravity, but rather the efforts of the cilia. From the nasal cavity, the mucus can be removed from the body through either through the nose, throat or mouth.

The ostia are very small, and can easily become blocked, preventing the normal drainage of mucus from the sinuses. This often occurs due to the inflammation produced by colds or allergy (an exaggerated reaction of the immune system to certain foreign invaders that is mistaken as a threat to the body), excessive mucus production or growths such as polyps (bulging growths that develop in the lining of mucous membranes). After the ostia are blocked, an inflammation or an infection can occur in the sinuses. This condition is called sinusitis. The air trapped in the sinuses during this condition can cause painful facial pressure, headaches and even toothaches.

The ostia openings, mucus production and cilia are all interrelated. If one of the three mechanisms malfunctions, a cycle of problems can develop that often leads to a sinus problem as an infection. This cycle can be difficult to break after it has started.

In some cases, problems may occur with the nose that can impact a persons' health. For example, allergies (exaggerated reactions of the immune system to certain foreign invaders that are mistaken as a threat to the body), the common colds and other disorders may lead to nasal symptoms such as congestion, sneezing, runny nose and thick nasal discharge. This can increase the risk of health problems such as asthma (a chronic inflammation of the airway tissues) and bronchitis (an inflammation of the mucous membranes in the bronchial tubes, which connect the windpipe to the lungs) and can lead to several other problems, including reduced sense of smell, dry mouth as a result of excessive mouth breathing, bad breath, snoring, noisy breathing and increased ingestion of pollution and germs as a result of mouth breathing. Meanwhile, sinus problems can be related to many problems with the nose, including inflammation, infection and allergic reactions. In many cases, such problems can lead to sinusitis, a condition that occurs when the lining of the sinuses becomes inflamed. Anything that triggers swelling or keeps the cilia (tiny, hair-like projections) from moving mucus (a substance secreted by mucous membranes) can lead to sinusitis. This includes factors such as temperature shifts, changes in air pressure, overuse of decongestant nasal sprays (drugs that reduce nasal congestion by narrowing the blood vessels in the membranes lining the nose), and smoking, swimming and diving. In addition, polyps (bulging growths that develop in the lining of mucous membranes) that block the sinus passage can cause sinusitis. When bacterial or viral infections cause sinusitis, the result is a sinus infection.

### SUMMARY OF THE INVENTION

The invention is a method and therapeutic apparatus for normalizing function of the sinus cilia using heat. It wraps around the head in a method similar to sleep shades, but the apparatus includes a heated element that sits on the face above the primary sinus cavities in the human head. The invention provides a non-surgical, non-invasive, drug-free therapeutic apparatus and method, of using thermal energy-in-transfer to normalize poor sinus cilia function that can cause upper respiratory problems. The heated element is inserted by the user into a pocket on the device, and it is removed after use. The heated element can be either a disposable packet that draws heat from air activated heat packets, gel packs, microwavable bean bags, instant chemical packs, hot water bottles, electric heating pads or silica hot packs. Most importantly, the heat pack must provide a temperature of the packet up to 140 degrees Fahrenheit for up to 18 hours. As contained heat pack is held in place above the sinus cavities on the face with a band that circle around the head, the sinus cavities and cilia within are heated beyond normothermia closer to 104 degrees Fahrenheit which optimizes cilia performance.

The nasal and sinus cavity membranes have thousands of very small hair-like organelles called cilia. They are capable of rhythmic beating like oars, and they keep the sinus cavities clear and clean from debris, pollutants and excessive drainage. When they are pulsating according to their normal rhythm, they move the bacteria from front to back of the nose. This system is called The Mucociliary Clearance system and consists of the cells of the surface of the nose, the cilia, and the mucus. They operate best if brought to a temperature slightly above normal human body temperature, also known as normothermia. If the Mucociliary Clearance system is performing abnormally, problems can include: sinus irritation, sinus infection, or sinusitis which may include inflammation of the sinuses and nasal passages, headache or pressure in the eyes, nose, and cheek area or on one side of the head. A person with a sinus problem may also have coughs and fever, bad breath, and nasal congestion with thick nasal secretions. The apparatus and method includes a mask with a pocket cavity that accommodates the heating element. When the apparatus is affixed to the human head with the heating element engaged, the warmth helps to restore normal cilia function by bringing the temperature of the sinus cavities to a more optimal temperature which is above normothermia.

### DESCRIPTION OF DRAWINGS

Drawings in a separate file.

### LIST OF REFERENCE NUMBERS

1.) N/A
2.) Entire invention
3.) N/A
4.) Outer facing fabric of pouch
5.) Ethmoid sinuses
6.) Frontal sinuses
7.) Maxillary sinuses
8.) Heating pack
9.) Inner facing fabric of pouch (proximal to face)
10.) Insertion opening for heat pack
11.) Right-side elastic strap
12.) Left-side elastic strap
13.) Right-side Velcro attachment
14.) Left-side Velcro attachment
15.) Outermost edge of right-side strap
16.) Outermost edge of left-side strap
17.) Right-side insertion opening for heating pack type-3
18.) Left-side insertion opening for heating pack type-3
19.) Heating pack type-2
20.) Heating pack type-3
21.) Curvature of invention to fit around nose
22.) Right-side clasp
23.) Skin/bone
24.) Extending fabric fibers for trapping hot air
25.) Left-side clasp

### DESCRIPTION OF THE ILLUSTRATIONS

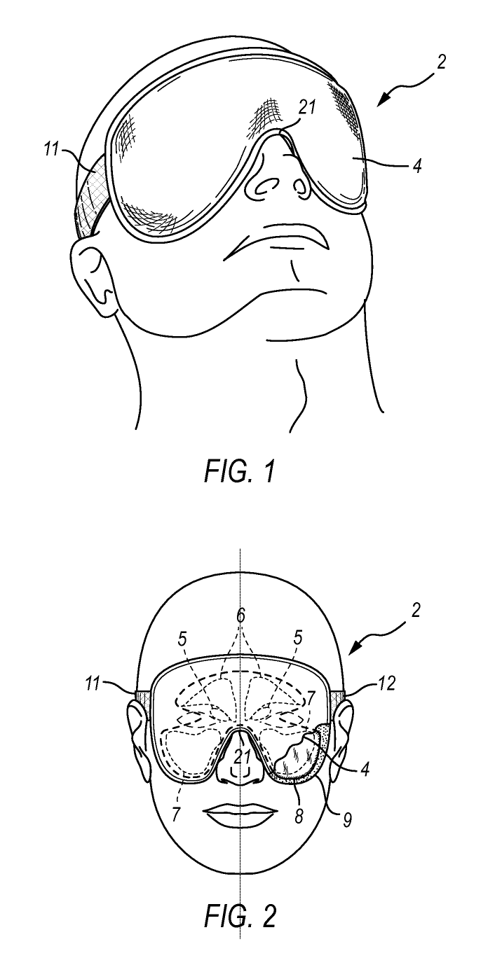

FIG. 1 is a perspective right side view of the user wearing the invention.

FIG. 2 is an elevation of the user and invention in FIG. 1 which includes the relative location of the sinus cavities.

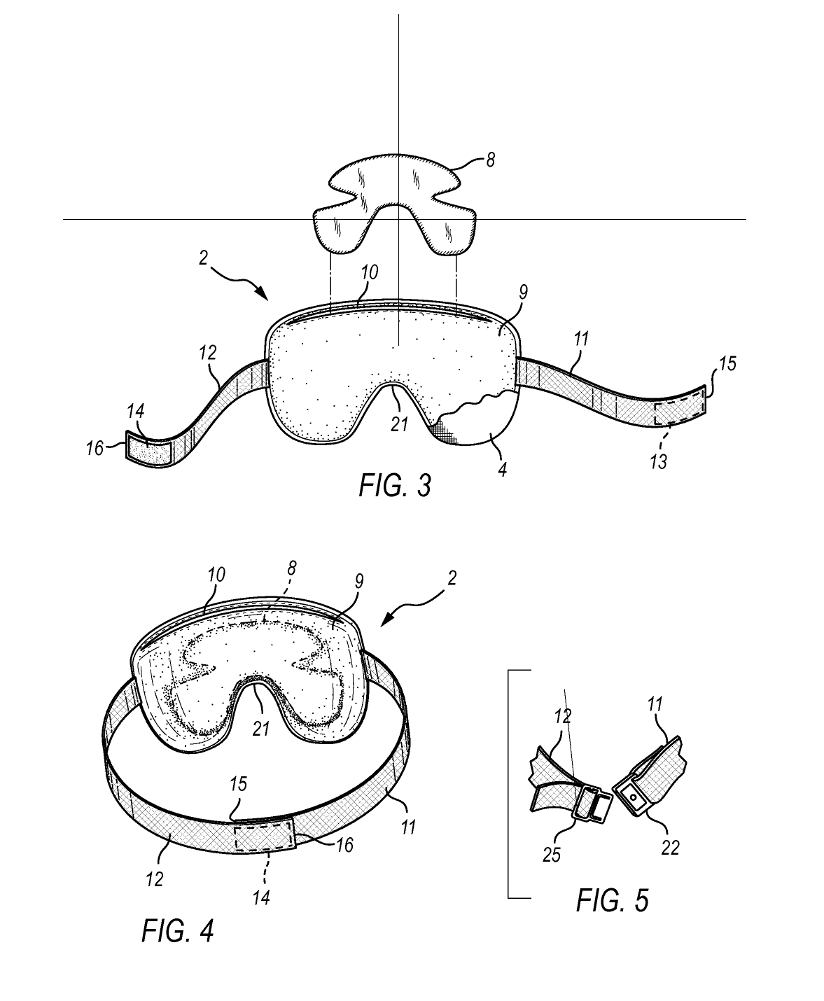

FIG. 3 is a perspective showing the side of the invention that is proximal to the face of the user as it appears when the heat pack is being inserted.

FIG. 4 is a perspective showing the side of the invention that is proximal to the face of the user which shows location of the straps for attachment as they are connected.

FIG. 5 is a perspective of an alternative clasp attachment.

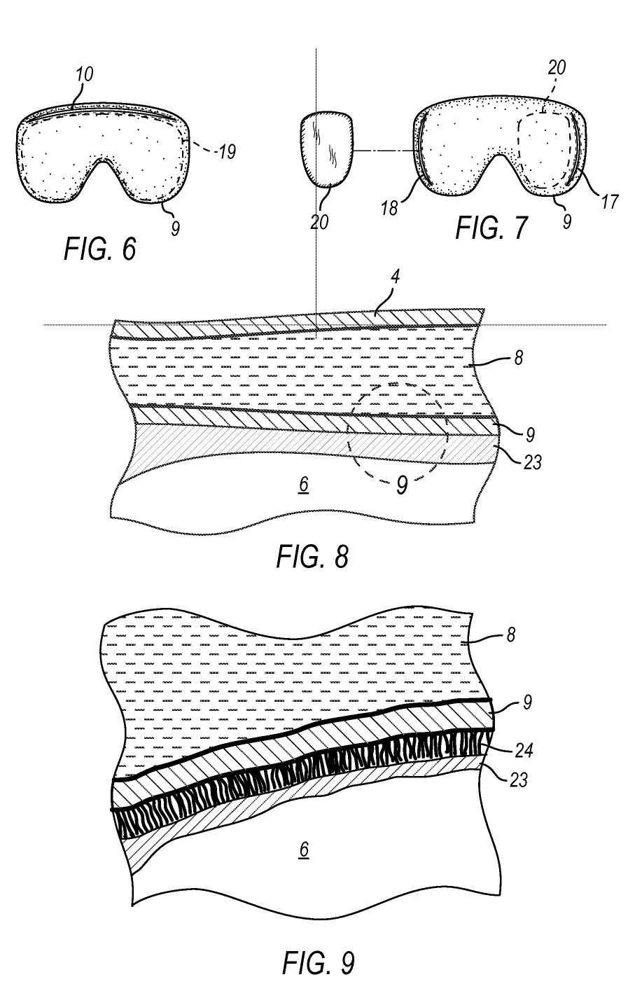

FIG. 6 is a partial elevation of the side of the invention that is proximal to the face which shows a heat pack that conforms to the size of the pouch.

FIG. 7 is a partial elevation of the side of the invention that is proximal to the face which shows alternative heat pack insertion openings as well as multiple heat packs.

FIG. 8 is a sectional view through pouch, heat pack, and user's face and sinus cavities.

FIG. 9 is a detailed sectional view of FIG. 8.

### DETAILED DESCRIPTION OF THE INVENTION

The following detailed description should be read with reference to the illustrations or drawings. The drawings should not necessarily be scaled with embodiments which were not created to limit the scope of the invention.

Embodiments of the present invention include an apparatus and a method that is made useful for normalizing sinus cilia function. The method along with the apparatus could be used as a relief for those who suffer from abnormal sinus cilia function. The method used is a thermal energy in-transfer which normalizes poor sinus cilia function that can also cause upper respiratory track problems.

The apparatus used, in one embodiment, is composed of a garment appliance that is wrapped around the human face. The said garment article is flexible and sufficiently supple to conform to the face and head's contours. The garment is also composed of both inner and outer adjacent layer of cloth which forms the pouch for receiving and supporting the heating packet therein. The pouch has an opening which enables the heating packet/element to be inserted into it.

In another embodiment, the heated element is inserted into a pocket on the device and is removed after use. It may vary from either a disposable packet that is able to draw heat from air activated heat packets, gel packs, microwavable bean bags, instant chemical packs, hot water bottles, electric heating pads or silica hot packs. The heat packs which are used must be able to provide a temperature of up to 140 degrees to the packet for up to 18 hours. With this, the sinus cavities and cilia are heated beyond normothermia closer to about 104 degrees Fahrenheit which then optimizes cilia performance.

The inner layer of the cloth pouch is composed of a porous layer positioned between the heating packet and the person's head and face. This compromises a cloth including a multiplicity of fibers extending from a portion of the cloth for holding trapped air to control heat distribution by diffusing the heat supplied by the heating packet to the face to cause the cilia to heat up to a more optimal temperature that is above 98.6 degrees Fahrenheit.

The method in the embodiments includes attaching a mask with a pocket cavity which accommodates the heating element. When the said apparatus is affixed to the human head with the heating element engaged, the warmth from the apparatus helps restore normal function of the sinus cilia by bringing the temperature of the sinus cavities to a more optimal temperature, otherwise known as above normothermia.

This method is more convenient as compared to the use of water-based irrigators or sinus douches which has side effects associated with water shooting up the nostrils. In addition, it is inconvenient to use water for heat transfer via the sinus passages. The water along with its associated pressure may be harsh to the sinuses.

What is claimed is:

1. A garment and appliance that is wrapped around the human head which includes a pocket in front of the face for a heating element that can warm the wearer's sinus cilia to a temperature that is higher than normothermia,

2. The garment article as stated in claim 1 is formed from a cloth for covering a portion of the body, particularly the face. The said garment article is flexible and sufficiently supple to conform to the face and head's contours,

3. The said garment article in claim 1 is composed of an inner and outer adjacent layer of cloth forming a pouch for receiving and supporting the flexible heating packet therein,

4. The pouch as stated in claim 3 has an opening to enable the heating packet to be inserted into it before use and removed after use,

5. The pouch as stated in claim 3 is elongated to provide a passage that is substantially longer than the width of the heating packet and extends in a general shape to mirror the location of the underlying sinus cavities in the human head,

6. The said flexible heating packet as stated in claim 3 contains substances that provide either an air or a chemical reaction wherein it can be heated to achieve a temperature up to 140 degrees Fahrenheit for up to eighteen (18) hours,

7. The inner layer of the cloth pouch as stated in claim 3 is composed of a porous layer positioned between the heating packet and the person's face. This consists of a cloth including a multiplicity of fibers extending from a portion of the cloth for holding trapped air to control heat distribution by diffusing the heat supplied by the heating packet to the face to cause the cilia to heat up to a more optimal temperature that is above 98.6 degrees Fahrenheit,

8. The heating packet as stated in claim 3 is smaller than the pouch which extends substantially from one end of the garment to the other end with the elongated shape of the pouch serving as a means for enabling the relatively smaller heating packet to be shifted within the garment to slightly different positions. The friction between the pouch and the packet holds the heating packet in the desired position.

9. A garment in combination with an inserted heating packet includes a cloth head covering with left and right covering for the sides of the head so as to stay firmly on the head using an elastic fastener around the head and keeping it positioned on the head above the sinus cavities keeping them covered for heat transference.
